(19) United States
(12) Patent Application Publication
(10) Pub. No.: US 2025/0241624 A1
(43) Pub. Date: Jul. 31, 2025

(54) ULTRASOUND DIAGNOSTIC APPARATUS AND ULTRASOUND DIAGNOSTIC PROGRAM

(71) Applicant: FUJIFILM Corporation, Tokyo (JP)

(72) Inventors: Hideki YOSHIKAWA, Tokyo (JP); Yushi TSUBOTA, Tokyo (JP); Koji WAKI, Tokyo (JP)

(73) Assignee: FUJIFILM Corporation, Tokyo (JP)

(21) Appl. No.: 19/037,850

(22) Filed: Jan. 27, 2025

(30) Foreign Application Priority Data
Jan. 31, 2024 (JP) 2024-013367

(51) Int. Cl. A61B 8/00

(52) U.S. Cl. CPC A61B 8/5246; A61B 8/5223

(57) ABSTRACT

A irregularity analysis unit executes feature point extraction processing of extracting a plurality of feature points for each of a plurality of B-mode images acquired at different positions, and evaluation processing of generating information indicating irregularity of a tissue based on the feature points extracted for each of the plurality of B-mode images. The evaluation processing includes processing of executing distance evaluation processing of obtaining a feature point distance between a feature point of one B-mode image among two B-mode images and a feature point of the other B-mode image for each feature point in each of the two B-mode images, for each image pair including a reference B-mode image among the plurality of B-mode images and each of the other plurality of B-mode images, and processing of generating the information indicating the irregularity based on the feature point distance obtained for each feature point in each image pair.

### CROSS-REFERENCE TO RELATED APPLICATIONS

This application claims benefit of priority to Japanese Patent Application No. 2024-013367 filed Jan. 31, 2024, the entire contents of which are incorporated herein by reference.

### BACKGROUND OF THE INVENTION

### 1. Field of the Invention

The present disclosure relates to an ultrasound diagnostic apparatus and an ultrasound diagnostic program, and particularly to evaluation of a tissue in a subject.

### 2. Description of the Related Art

The ultrasound diagnostic apparatus is used to determine whether a tumor is benign or malignant. In general, the determination of whether the tumor is benign or malignant is manually performed by a doctor with reference to an ultrasound image such as a B-mode image acquired by the ultrasound diagnostic apparatus. JP2020-54815A discloses that hemangioma is diagnosed by using a plurality of ultrasound images acquired in time series.

### SUMMARY OF THE INVENTION

A malignant tumor has a feature that the irregularity of an internal structure is higher than that of a benign tumor. In view of this feature, it is considered to determine whether the tumor is benign or malignant by using the ultrasound diagnostic apparatus. However, the doctor is required to have a lot of experience and is required to make a lot of effort. Therefore, there is a demand for a technique that enables easy determination of the tumor is benign or malignant by using the ultrasound diagnostic apparatus.

An object of the present disclosure is to provide an ultrasound diagnostic apparatus and an ultrasound diagnostic program which facilitate evaluation of a tissue of a subject.

An aspect of the present disclosure relates to an ultrasound diagnostic apparatus comprising: an information processing unit that executes feature point extraction processing of extracting a plurality of feature points for each of a plurality of ultrasound images acquired at different positions in a subject, and evaluation processing of generating information indicating irregularity of a tissue in the subject based on the feature points extracted for each of the plurality of ultrasound images.

In the aspect, the evaluation processing includes processing of executing distance evaluation processing of obtaining a feature point distance between a feature point of one ultrasound image among two ultrasound images and a feature point of the other ultrasound image corresponding to the feature point of the one ultrasound image for each feature point in each of the two ultrasound images, for each image pair including a reference ultrasound image, which is any one of the plurality of ultrasound images, and each of the other plurality of ultrasound images, and processing of generating the information indicating the irregularity based on the feature point distance obtained for each feature point in each image pair.

In the aspect, the distance evaluation processing includes processing of obtaining a deviation, from the reference ultrasound image that is one of the image pair, of the other ultrasound image, and processing of obtaining the feature point distance after correcting a position of the feature point of the other ultrasound image by the obtained deviation.

In the aspect, the information processing unit determines a color for each feature point in the reference ultrasound image based on the feature point distance obtained for each image pair, and generates an image in which a local region corresponding to each feature point in the reference ultrasound image is colored.

In the aspect, the evaluation processing includes processing of executing feature point exterior shape evaluation processing of obtaining a feature point exterior shape defined by the plurality of feature points for each of the plurality of ultrasound images, and obtaining difference information between one feature point exterior shape and the other feature point exterior shape for each image pair including a reference ultrasound image, which is any one of the plurality of ultrasound images, and each of the other plurality of ultrasound images, and processing of generating the information indicating the irregularity based on the difference information obtained for each image pair.

In the aspect, the feature point exterior shape evaluation processing includes processing of obtaining a deviation, from the reference ultrasound image that is one of the image pair, of the other ultrasound image, and processing of obtaining the difference information after correcting a position of the other feature point exterior shape by the obtained deviation.

In the aspect, the feature point is a point at which a feature value indicating a geometric feature appearing in the ultrasound image is maximized or a point at which the feature value exceeds a predetermined threshold value.

Another aspect of the present disclosure relates to a processor-readable recording medium storing an ultrasound diagnostic program, the ultrasound diagnostic program causing a processor to execute: feature point extraction processing of extracting a plurality of feature points for each of a plurality of ultrasound images acquired at different positions in a subject, and evaluation processing of generating information indicating irregularity of a tissue in the subject based on the feature points extracted for each of the plurality of ultrasound images.

In the other aspect, the evaluation processing includes processing of executing distance evaluation processing of obtaining a feature point distance between a feature point of one ultrasound image among two ultrasound images and a feature point of the other ultrasound image corresponding to the feature point of the one ultrasound image for each feature point in each of the two ultrasound images, for each image pair including a reference ultrasound image, which is any one of the plurality of ultrasound images, and each of the other plurality of ultrasound images, and processing of generating the information indicating the irregularity based on the feature point distance obtained for each feature point in each image pair.

In the other aspect, the evaluation processing includes processing of executing feature point exterior shape evaluation processing of obtaining a feature point exterior shape defined by the plurality of feature points for each of the plurality of ultrasound images, and obtaining difference information between one feature point exterior shape and the other feature point exterior shape for each image pair including a reference ultrasound image, which is any one of the plurality of ultrasound images, and each of the other plurality of ultrasound images, and processing of generating the information indicating the irregularity based on the difference information obtained for each image pair.

According to the aspects of the present disclosure, the evaluation of the tissue of the subject can be facilitated.

### BRIEF DESCRIPTION OF THE DRAWINGS

FIG. 1 is a diagram showing a configuration of an ultrasound diagnostic apparatus according to an embodiment of the present disclosure.

FIG. 2 is a diagram showing analysis target data.

FIG. 3 is a diagram showing processing of obtaining a feature point evaluation value.

FIG. 4 is a flowchart of the processing of obtaining the feature point evaluation value.

FIG. 5 is a diagram showing a color map image.

FIG. 6 is a diagram showing processing in a case in which pieces of analysis image data are sequentially generated over time.

FIG. 7 is a diagram showing an example of processing of obtaining a feature point exterior shape for each B-mode image data.

FIG. 8 is a flowchart of processing of generating the analysis image data based on the feature point exterior shape obtained for each of pieces of B-mode image data of n frames.

FIG. 9 is a diagram showing that irregularity index determination processing is executed for reference B-mode image data and each of pieces of B-mode image data of a second frame to an n-th frame.

FIG. 10 is a diagram showing a feature point exterior shape obtained for reference B-mode image data and a feature point exterior shape obtained for B-mode image data of another frame.

FIG. 11 is a diagram showing an example of processing of obtaining an evaluation area of the feature point exterior shape for each B-mode image data.

FIG. 12 is a diagram showing correction processing for feature points.

### DESCRIPTION OF THE PREFERRED EMBODIMENTS

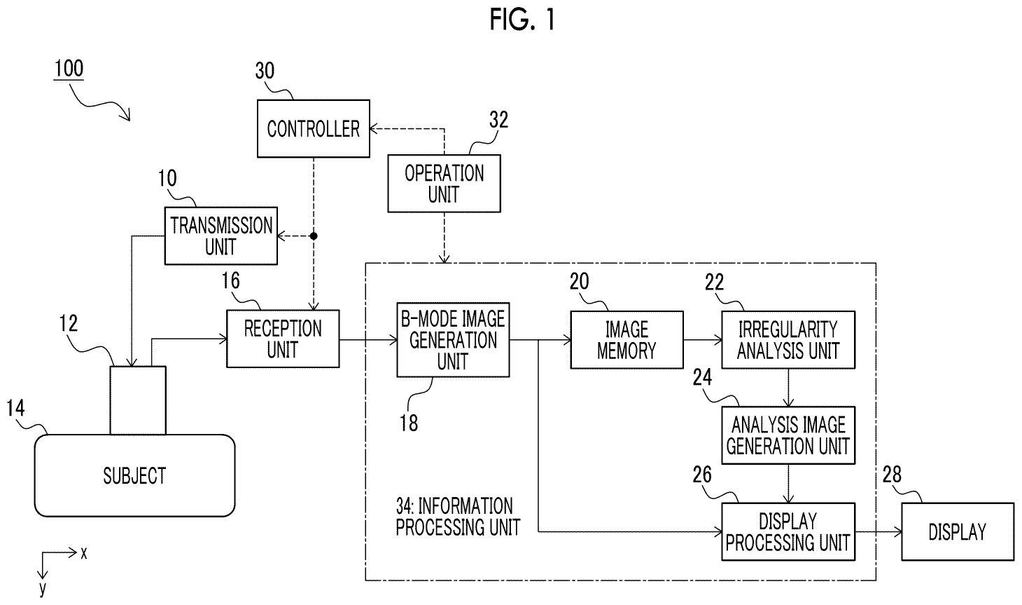

An embodiment of the present disclosure will be described with reference to the respective drawings. The same components shown in a plurality of drawings are denoted by the same reference numerals, and the description thereof will not be repeated. FIG. 1 shows a configuration of an ultrasound diagnostic apparatus 100 according to the embodiment of the present disclosure. The ultrasound diagnostic apparatus 100 comprises a transmission unit 10, an ultrasound probe 12, a reception unit 16, an information processing unit 34, a display 28, a controller 30, and an operation unit 32. The operation unit 32 may comprise a button, a lever, a keyboard, a mouse, and the like. The operation unit 32 may be a touch panel provided on the display 28. The controller 30 performs overall control of the ultrasound diagnostic apparatus 100 in accordance with an operation of the operation unit 32 performed by a user.

The ultrasound probe 12 comprises a plurality of transducers. The transmission unit 10 outputs a transmission signal to each transducer. Each transducer converts the transmission signal into ultrasonic waves and transmits the ultrasonic waves to a subject 14. The transmission unit 10 adjusts a delay time of the transmission signal to be output to each transducer such that the ultrasonic waves emitted from the respective transducer reinforce each other in a specific direction. As a result, a transmission beam caused by the ultrasonic wave is formed in the specific direction. The transmission unit 10 changes the delay time of the transmission signal to be output to each transducer, to scan the subject 14 with the transmission beam.

Each of the plurality of transducers receives the ultrasonic wave reflected by the subject 14, converts the received ultrasonic wave into an electric signal, and outputs the electric signal to the reception unit 16. The reception unit 16 generates a reception signal by phase-adding the electric signals output from the respective transducers such that the electric signals based on the ultrasonic waves received from a direction of the transmission beam reinforce each other, and outputs the reception signal to the information processing unit 34. A reception beam is formed in the ultrasound probe 12 by the phase-addition. Here, the reception beam refers to a directional pattern indicating a direction in which the ultrasonic waves, in which the reception signals reinforce each other, arrive. The reception signal corresponding to the reception beam is output from the reception unit 16 to the information processing unit 34, as a signal for generating B-mode image data. In the following description, the transmission beam and the reception beam will be collectively referred to as transmission/reception beams.

The information processing unit 34 comprises a B-mode image generation unit 18, an image memory 20, an irregularity analysis unit 22, an analysis image generation unit 24, and a display processing unit 26. The information processing unit 34 may include a processor that implements a function of each component by executing a program. In this case, the processor executes the program to configure each component (B-mode image generation unit 18, image memory 20, irregularity analysis unit 22, analysis image generation unit 24, and display processing unit 26).

The reception signal output from the reception unit 16 is input to the B-mode image generation unit 18. The B-mode image generation unit 18 generates the B-mode image data based on the reception signal obtained in each scanning direction of the transmission/reception beams.

The transmission unit 10, the ultrasound probe 12, and the reception unit 16 repeatedly scan the subject 14 with the transmission/reception beams. The B-mode image generation unit 18 sequentially generates pieces of B-mode image data over time at a predetermined frame rate.

The ultrasound diagnostic apparatus 100 may operate in any mode of a B-mode or an irregularity analysis mode described later.

In the operation in the B-mode, the display processing unit 26 generates video signals indicating B-mode images that are sequentially generated over time based on the pieces of B-mode image data that are sequentially generated over time, and outputs the video signals to the display 28. In this case, the display 28 displays an image based on the B-mode images that are sequentially generated over time, that is, a real-time B-mode image, based on the video signals.

In the operation in the irregularity analysis mode, the ultrasound diagnostic apparatus 100 stores the B-mode image data that are sequentially acquired over time, and executes irregularity analysis processing of analyzing the irregularity of a tissue in the subject 14. In the irregularity analysis processing, the ultrasound probe 12 is transported along a surface of the subject 14. The ultrasound probe 12 may be transported by the user who transports the ultrasound probe 12 using hand. A mechanism configured for transport may be used. A transport direction of the ultrasound probe 12 is a direction intersecting a surface scanned with the transmission/reception beams, that is, an observation surface for which the B-mode image is acquired. The transport direction of the ultrasound probe 12 may be a direction perpendicular to the observation surface. The series of pieces of B-mode image data (hereinafter, referred to as analysis target data) that are sequentially generated by the B-mode image generation unit 18 over time are stored in the image memory 20. The analysis target data stored in the image memory 20 is a target of the irregularity analysis processing.

In the image memory 20, a region for storing the pieces of B-mode image data of n frames as the analysis target data may be secured. In this case, in a case in which the B-mode images stored in the image memory 20 have reached n frames and a new B-mode image is generated, the oldest B-mode image data may be deleted from the image memory 20, and then the new B-mode image may be stored in the image memory 20.

The irregularity analysis unit 22 executes the irregularity analysis processing on the analysis target data stored in the image memory 20, to generate irregularity information. The irregularity information is information indicating the irregularity of the tissue for which the analysis target data is acquired. The analysis image generation unit 24 generates the analysis image data indicating the irregularity of the tissue, and outputs the analysis image data to the display processing unit 26. The display processing unit 26 generates the video signal indicating an analysis image based on the analysis image data, and outputs the video signal to the display 28. The display 28 displays the analysis image based on the video signals.

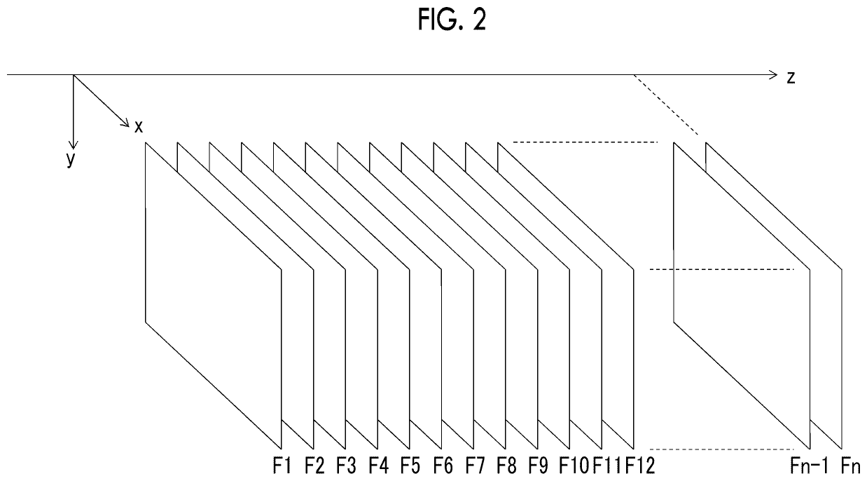

The irregularity analysis processing will be described in detail. FIG. 2 conceptually shows the analysis target data. The analysis target data is composed of the pieces of B-mode image data of n frames representing n B-mode images F1 to Fn. The analysis target data is generated in a case in which the ultrasound probe 12 is transported in the direction perpendicular to the observation surface. In the example shown in FIG. 2, the observation surface of each B-mode image is parallel to an xy plane, and the analysis target data indicates n B-mode images connected in a z-axis direction perpendicular to the xy plane. In the following description, the B-mode image data representing the B-mode images F1 to Fn may be referred to as the B-mode image data of a first frame to an n-th frame, respectively.

The irregularity analysis unit 22 sets a region of interest in the B-mode image for each of the pieces of B-mode image data of the n frames constituting the analysis target data, and obtains a distribution of a feature value in the region of interest. Here, the feature value refers to a value indicating a geometric feature appearing in the B-mode image. In the present embodiment, the feature value indicates a degree to which a corner of a linear image appears in the image. The feature value is larger in the corner of the linear image and the periphery thereof than in the surrounding region. As a method of obtaining the feature value, a Harris corner detection method or a minimum eigenvalue method is used.

The irregularity analysis unit 22 extracts feature points for each of the pieces of B-mode image data of the n frames constituting the analysis target data. Here, the feature point refers to a point at which the feature value is maximized or a point at which the feature value exceeds a predetermined threshold value. Extracting the feature point indicates obtaining position coordinates at which the feature point is present and acquiring the feature value at the feature point.

The irregularity analysis unit 22 selects the B-mode image data of one frame among the pieces of B-mode image data of the n frames constituting the analysis target data as reference B-mode image data. The reference B-mode image data may be, for example, initially acquired B-mode image data (B-mode image data of the first frame) having the smallest z-axis coordinate value. Hereinafter, an embodiment will be described in which the B-mode image data of the first frame is used as the reference B-mode image data, but the reference B-mode image data may be any B-mode image data selected from the second frame to the n-th frame.

The irregularity analysis unit 22 obtains a feature point distance between a plurality of feature points indicated by the reference B-mode image data and the corresponding feature points in the B-mode image data of each of the other frames. Here, the feature point corresponding to the feature point indicated by the reference B-mode image data refers to a feature point on the other B-mode image closest to the feature point on the reference B-mode image in a case in which the reference B-mode image and the other B-mode image are superimposed on each other. In addition, the feature point distance refers to a distance between the feature point on the reference B-mode image and the feature point on the other B-mode image corresponding to the feature point in a case in which the reference B-mode image and the other B-mode image are superimposed on each other. The irregularity analysis unit 22 further obtains, as a feature point evaluation value, an average value of the feature point distances obtained between the plurality of feature points indicated by the reference B-mode image data and the feature points in the B-mode image data of each of the other frames.

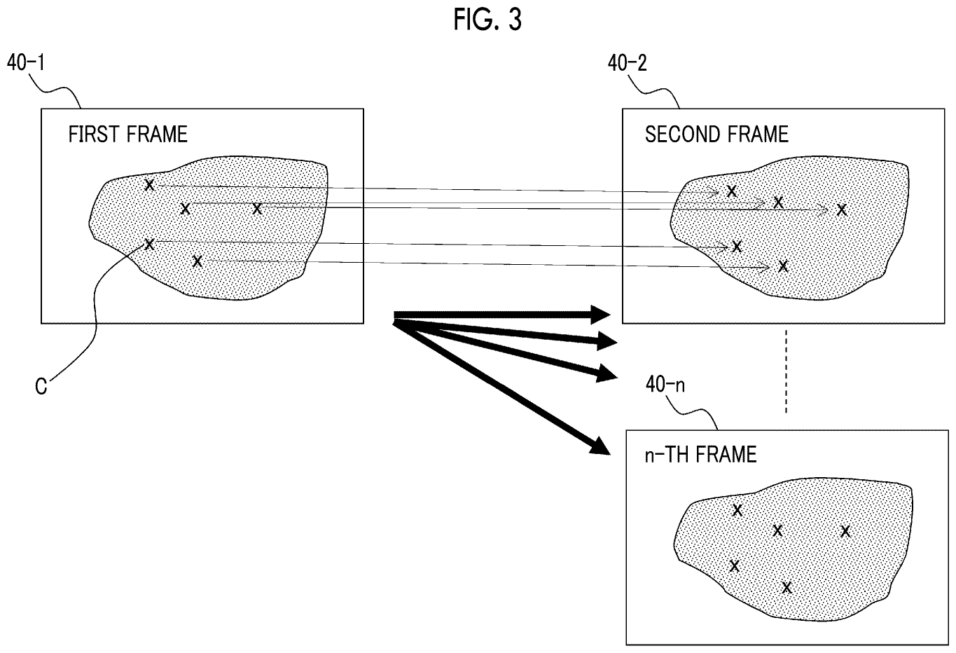

FIG. 3 conceptually shows processing of obtaining the feature point evaluation value. Five feature points C are shown in a reference B-mode image 40-1. An arrow toward the corresponding feature point in a B-mode image 40-2 of the second frame is drawn from each feature point C on the reference B-mode image 40-1. For the feature point C indicated by the reference B-mode image data, the feature point distance between the feature point C and the feature point on the B-mode image 40-2 of the second frame corresponding to the feature point C is obtained. The feature point distance for each feature point C on the reference B-mode image 40-1 is similarly obtained for each of a B-mode image 40-3 of the third frame to a B-mode image 40-n of the n-th frame. An average value of the feature point distances obtained for each of the second frame to the n-th frame is obtained as the feature point evaluation value for the feature point C.

It is assumed that k feature points are present on the reference B-mode image and that the number for identifying the feature points is j (j=1 to k). In addition, it is assumed that the number i for identifying the reference B-mode image is i=1 and the number i for identifying the other B-mode images are i=2 to n. A feature point distance between the j-th feature point on the reference B-mode image and the j-th feature point on the i-th B-mode image is denoted by d(j,i), and a feature point evaluation value Dj for the feature point j=1 to k is represented by (Expression 1). A symbol Σ in (Expression 1) indicates obtaining a total of addition for i=2 to n.

Dj=(1/(n−1))·Σd(j,i) (Expression 1)

The irregularity analysis unit 22 may obtain the average value of the feature point evaluation values Dj for j=1 to k as a tissue evaluation value D. The tissue evaluation value D indicates the irregularity of the tissue, and the tissue evaluation value D is larger as the irregularity is larger.

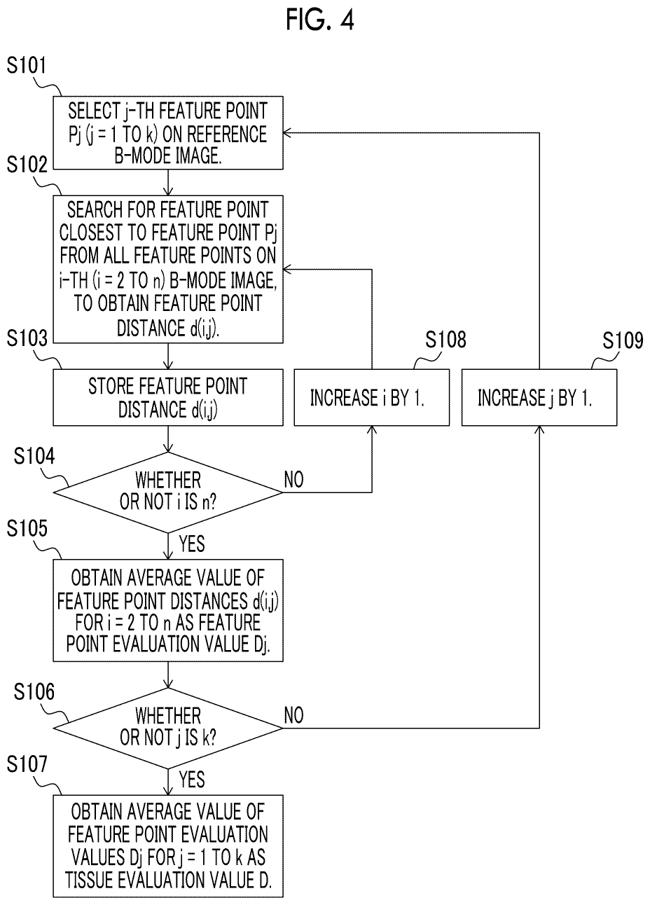

FIG. 4 is a flowchart of processing of obtaining the feature point evaluation value. In the flowchart, the B-mode image indicated by the B-mode image data of the i-th frame is simplified and represented as the i-th B-mode image. The irregularity analysis unit 22 selects a j-th feature point Pj (j=1 to k) from among the k feature points on the reference B-mode image (S101). Here, an initial value of j is 1.

The irregularity analysis unit 22 searches for the feature point closest to the feature point Pj from all the feature points on the i-th (i=2 to n) B-mode image, obtains the feature point distance d (i,j) (S102), and stores the feature point distance d (i,j) (S103). Here, an initial value of i is 2.

The irregularity analysis unit 22 determines whether or not i is n (S104), increases i by 1 in a case in which i is not n (S108), and returns to the processing of step S102. In a case in which i is n, the irregularity analysis unit 22 obtains the average value of the feature point distances d (i,j) for i=2 to n as the feature point evaluation value Dj (S105).

The irregularity analysis unit 22 determines whether or not j is k (S106), increases j by 1 in a case in which j is not k (S109), and returns to the processing of step S101. In a case in which j is k, the irregularity analysis unit 22 obtains the average value of the feature point evaluation values Dj for j=1 to k as the tissue evaluation value D (S107).

The irregularity analysis unit 22 outputs the irregularity information and the reference B-mode image data to the analysis image generation unit 24. Here, the irregularity information may include the feature point evaluation values Dj (j=1 to k) obtained for k feature points on the reference B-mode image, position information of the k feature points, the feature values of the k feature points, and the tissue evaluation value D. The analysis image generation unit 24 generates the analysis image data indicating the analysis image. The analysis image may be an image showing the B-mode image indicated by the reference B-mode image data, the position of each feature point, the feature value at each feature point on the reference B-mode image, the feature point evaluation value at each feature point on the reference B-mode image, the tissue evaluation value D, and the like.

The analysis image generation unit 24 outputs the analysis image data to the display processing unit 26. The display processing unit 26 generates the video signal indicating the analysis image based on the analysis image data, and outputs the video signal to the display 28. The display 28 displays the analysis image based on the video signals. The user may determine whether or not the tissue of the subject 14 is irregular with reference to the analysis image displayed on the display 28. Further, whether or not the subject 14 has a tumor or the like may be determined based on the determination. Further, in a case in which it is determined that there is the tumor, the user may determine whether the tumor is benign or malignant.

As described above, the irregularity analysis unit 22 executes the feature point extraction processing of extracting the plurality of feature points for each of the plurality of B-mode images (ultrasound images) acquired at different positions in the subject 14, and the evaluation processing of generating the information indicating the irregularity of the tissue in the subject 14 based on the feature points extracted for each of the plurality of B-mode images.

The evaluation processing includes processing of executing distance evaluation processing of obtaining the feature point distance between the feature point of one B-mode image among two B-mode images and the feature point of the other B-mode image corresponding to the feature point of the one B-mode image for each feature point in each of the two B-mode images, for each image pair including the reference B-mode image, which is any one of the plurality of B-mode images, and each of the other plurality of B-mode images, and processing of generating the information indicating the irregularity based on the feature point distance obtained for each feature point in each image pair.

An ultrasound diagnostic program for executing the feature point extraction processing and the evaluation processing may be loaded into the processor constituting the irregularity analysis unit 22 in the information processing unit 34. That is, the ultrasound diagnostic program causes the processor to execute the feature point extraction processing and the evaluation processing.

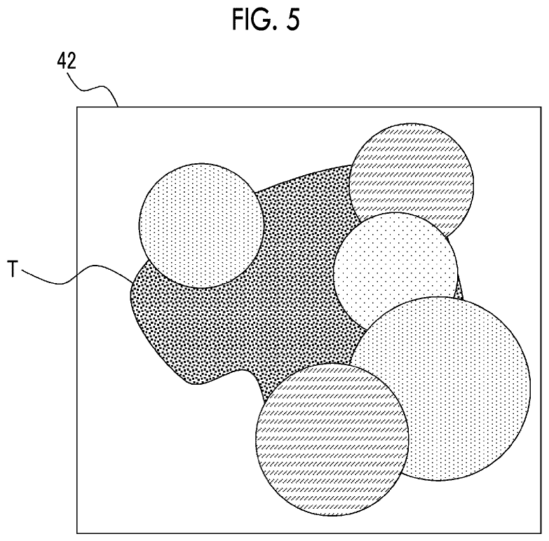

FIG. 5 shows a color map image 42 as an example of the analysis image. In this example, the position of each feature point and the feature point evaluation value are shown together with a tissue T by a circle centered on the position of each feature point. A radius of the circle indicates the feature point evaluation value. In addition, a color inside the circle may indicate the feature point evaluation value. For example, a color having a longer wavelength (color closer to red) is assigned to the inside of the circle as the feature point evaluation value is larger, and a color having a shorter wavelength (color closer to blue) is assigned to the inside of the circle as the feature point evaluation value is smaller. It should be noted that, instead of the display in which the radius of the circle is determined by the feature point evaluation value, the display may be performed in which the radius of the circle is determined by the feature value in the reference B-mode image.

As described above, the analysis image generation unit 24 determines a color for each feature point in the reference B-mode image (reference ultrasound image) based on the feature point distance obtained for each image pair, and generates an image in which the color is added to the inside (local region) of the circle corresponding to each feature point in the reference B-mode image.

The ultrasound diagnostic program loaded into the processor constituting the analysis image generation unit 24 causes the processor to execute processing of determining the color for each feature point in the reference B-mode image (reference ultrasound image) based on the feature point distance obtained for each image pair, and generating an image in which the color is applied to the inside (local region) of the circle corresponding to each feature point in the reference B-mode image.

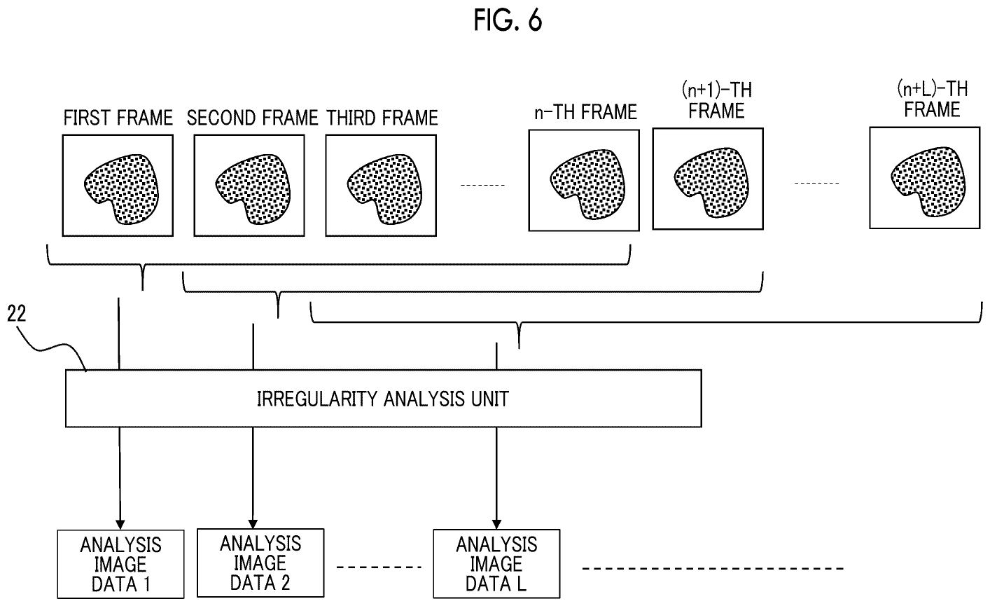

FIG. 6 conceptually shows processing in a case in which the pieces of analysis image data are sequentially generated over time. In a case in which the B-mode image data of the n-th frame is stored in the image memory 20, the irregularity analysis unit 22 executes the irregularity analysis processing on the pieces of B-mode image data from the first frame to the n-th frame stored by tracing back to the n−1 frames, and generates the analysis image data 1.

In a case in which the B-mode image data of the (n+1)-th frame is stored in the image memory 20, the irregularity analysis unit 22 generates analysis image data 2 based on the pieces of B-mode image data from the second frame to the (n+1)-th frame stored by tracing back to the n−1 frames. In a case in which the B-mode image data of the (n+L)-th frame is stored in the image memory 20, the irregularity analysis unit 22 generates analysis image data L based on the pieces of B-mode image data from the (1+L)-th frame to the (n+L)-th frame stored by tracing back to the n−1 frames.

As described above, each time the B-mode image data is newly stored in the image memory 20, the irregularity analysis unit 22 sequentially generates new analysis image data based on the series of the pieces of B-mode image data (analysis target data) of the n frames stored by tracking back from the B-mode image data to the n−1 frames.

Next, the irregularity analysis processing based on a feature point exterior shape will be described. The feature point exterior shape refers to a figure formed by connecting the closest feature points among the plurality of feature points on the B-mode image with a line.

The irregularity analysis unit 22 executes processing of extracting the feature points for each of the pieces of B-mode image data of the n frames constituting the analysis target data. The irregularity analysis unit 22 obtains feature point exterior shape data indicating the feature point exterior shape for each B-mode image data.

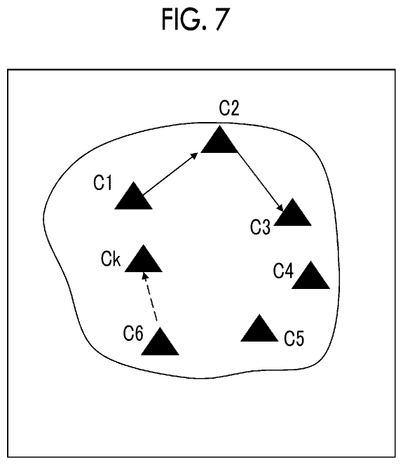

FIG. 7 conceptually shows an example of processing of obtaining the feature point exterior shape for each B-mode image data. The irregularity analysis unit 22 first searches for a first feature point C1 having the maximum feature value. The irregularity analysis unit 22 searches for a second feature point C2 closest to the first feature point C1 and obtains connection data representing a line connecting the first feature point C1 and the second feature point C2. It is assumed that the irregularity analysis unit 22 executes the same processing described later to search for a total of k feature points. The irregularity analysis unit 22 connects the first feature point C1 to a k-th feature point Ck in order and obtains connection data indicating each line connecting the k-th feature point Ck to the first feature point C1. The connection data indicating each line connecting the first feature point C1 to the k-th feature point Ck in order and connecting the k-th feature point Ck and the first feature point C1 is the feature point exterior shape data indicating the feature point exterior shape.

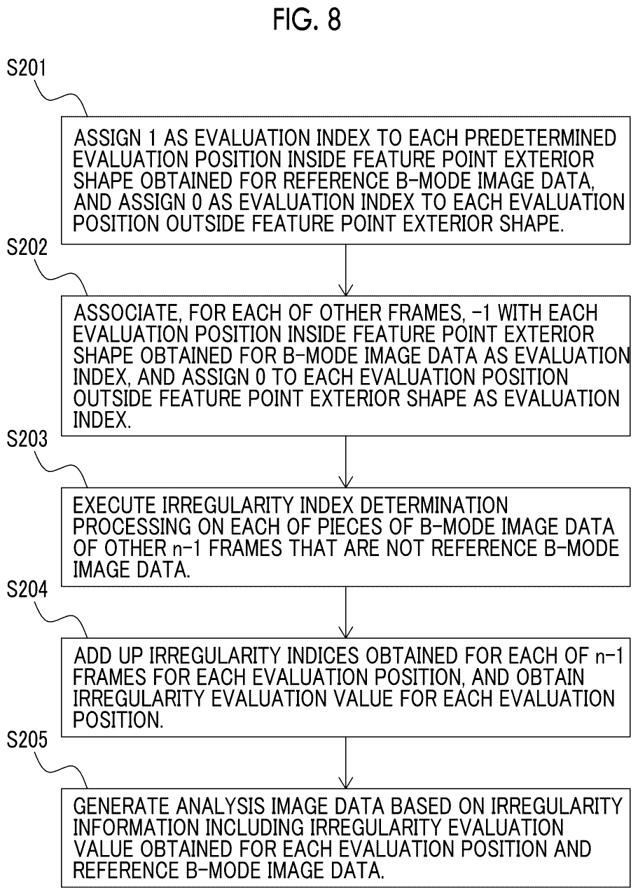

FIG. 8 shows a flowchart of processing of generating the analysis image data based on the feature point exterior shape obtained for each of the pieces of B-mode image data of the n frames. The irregularity analysis unit 22 assigns 1 as the evaluation index to each predetermined evaluation position inside the feature point exterior shape obtained for the reference B-mode image data, and assigns 0 as the evaluation index to each evaluation position outside the feature point exterior shape (S201).

In addition, for each of the other frames, the irregularity analysis unit 22 associates −1 with each evaluation position inside the feature point exterior shape obtained for the B-mode image data as the evaluation index, and assigns 0 to each evaluation position outside the feature point exterior shape as an evaluation index (S202).

The irregularity analysis unit 22 adds the evaluation index assigned to each evaluation position on the B-mode image for another frame to the evaluation index assigned to each evaluation position on the reference B-mode image for each evaluation position, and obtains an irregularity index for each evaluation position. The evaluation position at which the irregularity index is positive is in a region inside the feature point exterior shape on the reference B-mode image and outside the feature point exterior shape on the B-mode image for another frame. The evaluation position at which the irregularity index is negative is in a region outside the feature point exterior shape on the reference B-mode image and inside the feature point exterior shape on the B-mode image for another frame. The evaluation position at which the irregularity index is zero is in a region inside the feature point exterior shape on the reference B-mode image and inside the feature point exterior shape on the B-mode image for another frame. The irregularity analysis unit 22 executes irregularity index determination processing on each of the pieces of B-mode image data of the other n−1 frames that are not the reference B-mode image data (S203).

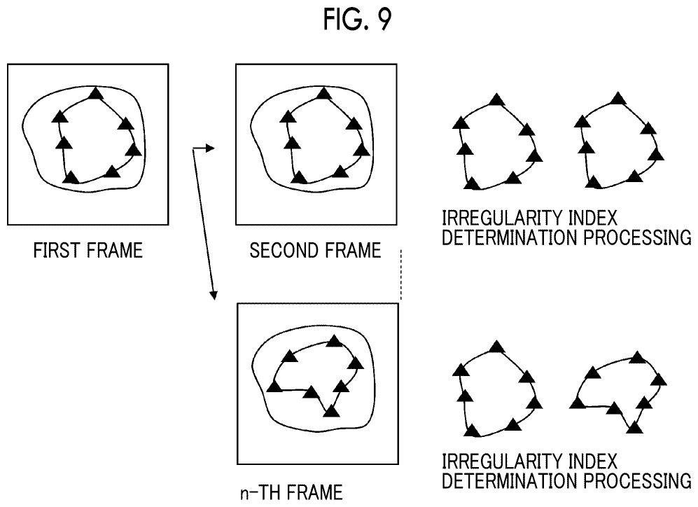

FIG. 9 conceptually shows that the irregularity index determination processing is executed for the B-mode image data of the first frame, that is, the reference B-mode image data, and each of the pieces of B-mode image data of the second frame to the n-th frame.

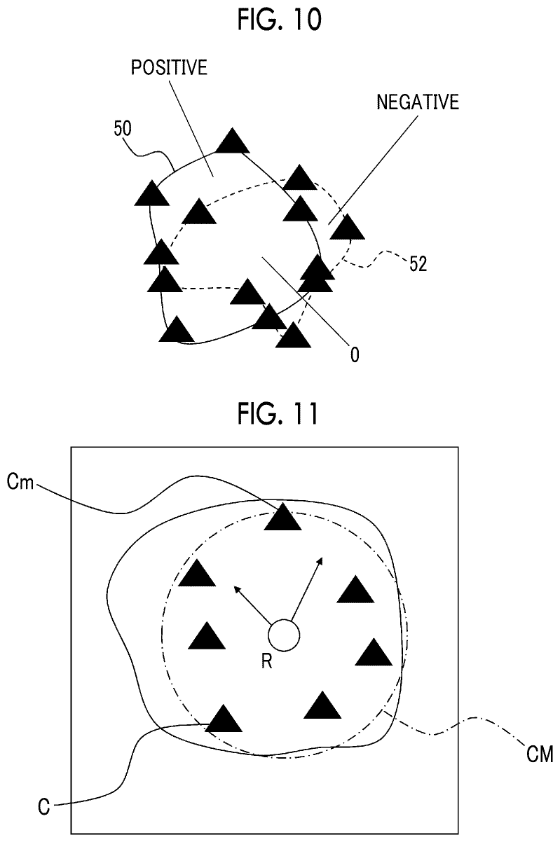

FIG. 10 conceptually shows a feature point exterior shape 50 obtained for the reference B-mode image data and a feature point exterior shape 52 obtained for the B-mode image data of another frame. At the evaluation position at which the irregularity index is positive, the feature point exterior shape is reduced in the B-mode image of the other frame as compared with the feature point exterior shape in the reference B-mode image. At the evaluation position at which the irregularity index is negative, the feature point exterior shape is enlarged in the B-mode image of the other frame as compared with the feature point exterior shape in the reference B-mode image. At the evaluation position where the irregularity index is zero, in the B-mode image of the other frame, the feature point exterior shape is neither enlarged nor reduced with respect to the feature point exterior shape in the reference B-mode image.

The irregularity index at each evaluation position can be referred to as difference information indicating a difference between the feature point exterior shape 50 obtained for the reference B-mode image data and the feature point exterior shape 52 obtained for the B-mode image data of another frame.

Returning to FIG. 8, the irregularity analysis unit 22 individually adds up the irregularity indices obtained for each of the n−1 frames for each evaluation position, and obtains an irregularity evaluation value for each evaluation position (S204).

The irregularity analysis unit 22 outputs the irregularity information including the irregularity evaluation value obtained for each evaluation position and the reference B-mode image data to the analysis image generation unit 24. The analysis image generation unit 24 generates the analysis image data indicating the analysis image (S205). The analysis image is an image in which each evaluation position on the B-mode image indicated by the reference B-mode image data and the periphery of each evaluation position are colored in accordance with the irregularity evaluation value. Here, the periphery of the evaluation position refers to, for example, a region in a circle determined by a predetermined radius centered on the evaluation position. Further, the periphery of the evaluation position may be a polygonal region including the evaluation position. As an absolute value of the irregularity evaluation value is larger, a color having a longer wavelength (color closer to red) is assigned, and as the absolute value of the irregularity evaluation value is smaller, a color having a shorter wavelength (color closer to blue) is assigned.

The irregularity analysis unit 22 may obtain the tissue evaluation value D indicating the irregularity of the biological tissue based on the analysis target data by the following processing. The irregularity analysis unit 22 executes a correlation operation on the feature point exterior shape obtained for the reference B-mode image data and the feature point exterior shape obtained for the B-mode image data of the other frame, to obtain a correlation value. The correlation value indicates a degree to which the two feature point exterior shapes are approximated. As the correlation value is larger, the two feature point exterior shapes are more approximated. The irregularity analysis unit 22 obtains, for example, the reciprocal of the average value of the correlation values obtained for each of the second frame to the n-th frame, as the tissue evaluation value D. The tissue evaluation value D may be obtained based on other relationships in which the tissue evaluation value D is larger as the average value of the correlation values is larger, in addition to the reciprocal of the average value of the correlation values. As the feature point exterior shape obtained for the reference B-mode image data and the feature point exterior shape obtained for the B-mode image data of each of the other frames are more different from each other, the average value of the correlation values is smaller, and the tissue evaluation value D is larger.

The irregularity analysis unit 22 outputs the irregularity information including the tissue evaluation value D to the analysis image generation unit 24. The analysis image generation unit 24 generates the analysis image data indicating the tissue evaluation value D and outputs the analysis image data to the display processing unit 26. The display processing unit 26 generates the video signal indicating the analysis image based on the analysis image data, and outputs the video signal to the display 28. The display 28 displays the analysis image based on the video signals.

It should be noted that the irregularity analysis unit 22 may obtain the tissue evaluation value D indicating the irregularity of the tissue by another processing described later. The irregularity analysis unit 22 obtains the feature point exterior shape for each of the B-mode image data of the n frames constituting the analysis target data, and obtains the evaluation area for the feature point exterior shape obtained for each B-mode image data.

FIG. 11 conceptually shows an example of processing of obtaining the evaluation area of the feature point exterior shape for the B-mode image data. The irregularity analysis unit 22 extracts the feature points C from each B-mode image data. The irregularity analysis unit 22 obtains coordinates of a representative point R of a region in which the plurality of extracted feature points C are present. An x coordinate value of the representative point R is, for example, a value obtained by adding an x coordinate value (x coordinate maximum value) of the feature point having the maximum x coordinate value among the plurality of feature points and an x coordinate value (x coordinate minimum value) of the feature point having the minimum x coordinate value among the plurality of feature points and dividing the sum by two. A y coordinate value of the representative point R is, for example, a value obtained by adding a y coordinate value (y coordinate maximum value) of the feature point having the maximum y coordinate value among the plurality of feature points and a y coordinate value (y coordinate minimum value) of the feature point having the minimum y coordinate value among the plurality of feature points and dividing the sum by two. The irregularity analysis unit 22 searches for a feature point Cm having the longest distance from the representative point R, and obtains an area of a circle CM having a radius of a distance between the representative point R and the feature point Cm searched for as the evaluation area.

The irregularity analysis unit 22 obtains an absolute value of a value obtained by subtracting the evaluation area obtained for the B-mode image data of the other frame from the evaluation area obtained for the reference B-mode image data, as an area difference value. The area difference value indicates a degree to which the two feature point exterior shapes are different from each other. As the area difference is larger, the two feature point exterior shapes are more different from each other. The irregularity analysis unit 22 obtains, for example, an average value of the area difference values obtained for each of the second frame to the n-th frame, as the tissue evaluation value D. As the evaluation area obtained for the reference B-mode image data and the evaluation area obtained for the B-mode image data of each of the other frames are more different from each other, the area difference value is larger, and the tissue evaluation value D is larger.

As described above, the evaluation processing executed by the irregularity analysis unit 22 includes first processing of executing feature point exterior shape evaluation processing of obtaining a feature point exterior shape defined by the plurality of feature points for each of the plurality of B-mode images (ultrasound images) and obtaining difference information between one feature point exterior shape and the other feature point exterior shape for each image pair including a reference B-mode image, which is any one of the plurality of B-mode images, and each of the other plurality of B-mode images, and second processing of generating the information indicating the irregularity based on the difference information obtained for each image pair. Here, the difference information includes the irregularity index, the correlation value, the area difference value, and the like.

The ultrasound diagnostic program loaded into the processor constituting the irregularity analysis unit 22 causes the processor to execute the first processing and the second processing.

In each of the above-described examples, it is assumed that there is no deviation in the position of the tissue appearing in the B-mode image of each frame. In the diagnosis in the irregularity analysis mode, the position of the tissue appearing in the B-mode image may be deviated due to camera shake or the like in the B-mode image of the first frame with respect to the B-mode image of the other frame. Therefore, the correction processing may be performed on the positions of the feature points for the second frame to the n-th frame in order to compensate for the deviation that has occurred at the position of the tissue.

The irregularity analysis unit 22 executes the correlation operation on the reference B-mode image while changing the position of the B-mode image of the other frame with respect to the reference B-mode image, obtains the position of the B-mode image of the other frame in which the correlation value is the maximum value, and obtains, as the positional deviation vector, an amount of positional deviation of the tissue appearing in the B-mode image of the other frame with respect to the tissue appearing in the reference B-mode image.

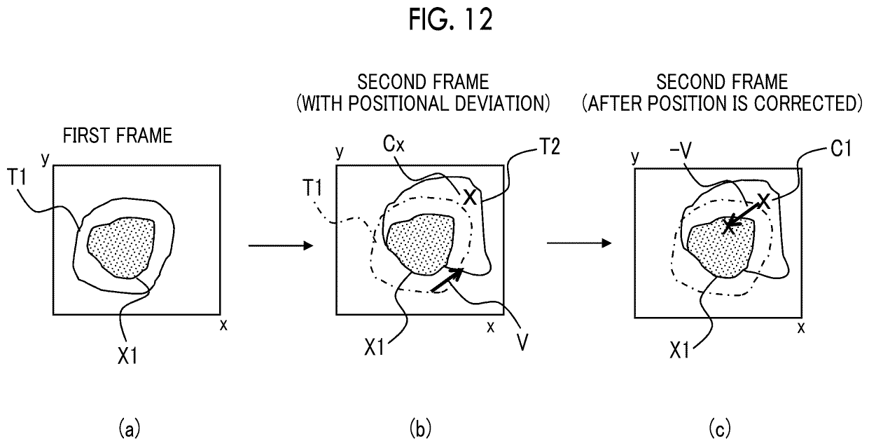

That is, the irregularity analysis unit 22 obtains the positional deviation vector for each of the second frame to the n-th frame. The irregularity analysis unit 22 subtracts the positional deviation vector obtained for each of the second frame to the n-th frame from the position coordinates of the feature point obtained for each of the second frame to the n-th frame. As a result, the irregularity analysis unit 22 obtains new position coordinates of the feature point obtained for each of the second frame to the n-th frame, to correct the position coordinates of each feature point.
(a) of FIG. 12 shows the reference B-mode image acquired in the xy plane. The reference B-mode image shows a region X1 in which a tissue T1 appears and the feature points extracted from the reference B-mode image are present. (b) of FIG. 12 shows a displaced tissue T2 and a feature point Cx extracted from the tissue T2 in the B-mode image of the second frame. In addition, a positional deviation vector V indicating a deviation of the tissue T2 with respect to the tissue T1 is shown.
(c) of FIG. 12 shows that the positional deviation vector Vis subtracted from the position coordinates of the feature point Cx set in the tissue T2, to correct the position coordinates of the feature point Cx.

The distance evaluation processing executed by the irregularity analysis unit 22 may include processing of obtaining a deviation of, from the reference B-mode image (reference ultrasound image) that is one of the image pair, of the other B-mode image, and first correction processing of obtaining the feature point distance after correcting a position of the feature point of the other ultrasound image by the obtained deviation. In addition, the feature point exterior shape evaluation processing may include processing of obtaining a deviation of, from the reference B-mode image (reference ultrasound image) that is one of the image pair, of the other B-mode image, and second correction processing of obtaining the difference information after correcting a position of the other feature point exterior shape by the obtained deviation.

The ultrasound diagnostic program loaded into the processor constituting the irregularity analysis unit 22 may cause the processor to execute the first correction processing and the second correction processing.

By correcting the position of the feature point in this way, even in a case in which the deviation occurs from the ideal linear transport trajectory in a case in which the ultrasound probe 12 is transported in the irregularity analysis mode, an appropriate position of the feature point is obtained.

What is claimed is:

1. An ultrasound diagnostic apparatus comprising:
an information processing unit that executes
feature point extraction processing of extracting a plurality of feature points for each of a plurality of ultrasound images acquired at different positions in a subject, and
evaluation processing of generating information indicating irregularity of a tissue in the subject based on the feature points extracted for each of the plurality of ultrasound images.

2. The ultrasound diagnostic apparatus according to claim 1,
wherein the evaluation processing includes
processing of executing distance evaluation processing of obtaining a feature point distance between a feature point of one ultrasound image among two ultrasound images and a feature point of the other ultrasound image corresponding to the feature point of the one ultrasound image for each feature point in each of the two ultrasound images, for each image pair including a reference ultrasound image, which is any one of the plurality of ultrasound images, and each of the other plurality of ultrasound images, and
processing of generating the information indicating the irregularity based on the feature point distance obtained for each feature point in each image pair.

3. The ultrasound diagnostic apparatus according to claim 2,
wherein the distance evaluation processing includes
processing of obtaining a deviation, from the reference ultrasound image that is one of the image pair, of the other ultrasound image, and processing of obtaining the feature point distance after correcting a position of the feature point of the other ultrasound image by the obtained deviation.

4. The ultrasound diagnostic apparatus according to claim 2,
wherein the information processing unit
determines a color for each feature point in the reference ultrasound image based on the feature point distance obtained for each image pair, and
generates an image in which a local region corresponding to each feature point in the reference ultrasound image is colored.

5. The ultrasound diagnostic apparatus according to claim 1,
wherein the evaluation processing includes
processing of executing feature point exterior shape evaluation processing of
obtaining a feature point exterior shape defined by the plurality of feature points for each of the plurality of ultrasound images, and
obtaining difference information between one feature point exterior shape and the other feature point exterior shape for each image pair including a reference ultrasound image, which is any one of the plurality of ultrasound images, and each of the other plurality of ultrasound images, and
processing of generating the information indicating the irregularity based on the difference information obtained for each image pair.

6. The ultrasound diagnostic apparatus according to claim 5,
wherein the feature point exterior shape evaluation processing includes
processing of obtaining a deviation, from the reference ultrasound image that is one of the image pair, of the other ultrasound image, and processing of obtaining the difference information after correcting a position of the other feature point exterior shape by the obtained deviation.

7. The ultrasound diagnostic apparatus according to claim 1,
wherein the feature point is a point at which a feature value indicating a geometric feature appearing in the ultrasound image is maximized or a point at which the feature value exceeds a predetermined threshold value.

8. A processor-readable recording medium storing an ultrasound diagnostic program, the ultrasound diagnostic program causing a processor to execute:
feature point extraction processing of extracting a plurality of feature points for each of a plurality of ultrasound images acquired at different positions in a subject, and
evaluation processing of generating information indicating irregularity of a tissue in the subject based on the feature points extracted for each of the plurality of ultrasound images.

9. The recording medium according to claim 8,
wherein the evaluation processing includes
processing of executing distance evaluation processing of obtaining a feature point distance between a feature point of one ultrasound image among two ultrasound images and a feature point of the other ultrasound image corresponding to the feature point of the one ultrasound image for each feature point in each of the two ultrasound images, for each image pair including a reference ultrasound image, which is any one of the plurality of ultrasound images, and each of the other plurality of ultrasound images, and
processing of generating the information indicating the irregularity based on the feature point distance obtained for each feature point in each image pair.

10. The recording medium according to claim 8,
wherein the evaluation processing includes
processing of executing feature point exterior shape evaluation processing of
obtaining a feature point exterior shape defined by the plurality of feature points for each of the plurality of ultrasound images, and
obtaining difference information between one feature point exterior shape and the other feature point exterior shape for each image pair including a reference ultrasound image, which is any one of the plurality of ultrasound images, and each of the other plurality of ultrasound images, and
processing of generating the information indicating the irregularity based on the difference information obtained for each image pair.
